(19) United States
(12) Patent Application Publication
(10) Pub. No.: US 2018/0036330 A1
(43) Pub. Date: Feb. 8, 2018

(54) NUCLEOTIDE PHOSPHORAMIDATE FORMULATION

(71) Applicant: MEDIVIR AB, Stockholm (SE)

(72) Inventor: Mattias ANDERSSON, Huddinge (SE)

(21) Appl. No.: 15/555,414

(22) PCT Filed: Mar. 2, 2016
(86) PCT No.: PCT/SE2016/050167

§ 371 Date: Sep. 1, 2017

(30) Foreign Application Priority Data
Mar. 2, 2015 (SE) 1550242-0
Mar. 4, 2015 (SE) 1550266-9

(51) Int. Cl. A61K 31/7072; A61K 9/48; A61K 45/06; A61K 47/10

(52) U.S. Cl. CPC A61K 31/7072; A61K 47/10; A61K 9/4858; A61K 45/06

(57) ABSTRACT

Compound 1 with the formula

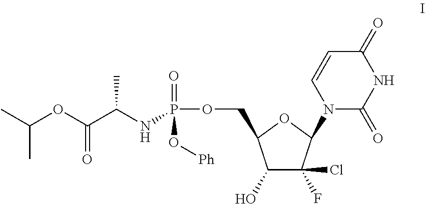 [SMILES: CC(C)OC(=O)[C@H](C)N[P@](=O)(OC[C@H]1O[C@@H](n2ccc(=O)[nH]c2=O)[C@@](F)(Cl)[C@@H]1O)Oc1ccccc1]

is an HCV antiviral protide, which is surprisingly soluble in ethanol, thereby facilitating the preparation of pharmaceutical formulations, such as adsorbed mesoporous carriers or SEDDS of Pouton Types III or IV.

### FIELD

The present application relates to galenic compositions useful in methods for the treatment of a disease condition such as a hepatitis C virus infection, liver fibrosis, and impaired liver function.

### BACKGROUND

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate, that 40% of chronic liver disease is HCV-related, resulting in an estimated 8,000-10.000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with using the standard of care (SOC) combination therapy a large percentage of patients fail therapy, i.e. are non-responders or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, a substantial increase in cirrhosis-related morbidity and mortality is likely to result among patients infected between the years of 1965-1985.

HCV is enveloped positive strand RNA virus in the Flaviviridae family. The single strand HCV RNA genome is approximately 9500 nucleotides m length and has a single open reading frame (ORF) encoding a single large polyprotein of about 3000 amino acids. In infected cells, this polyprotein is cleaved at multiple sites by cellular and viral proteases to produce the structural and non-structural (NS) proteins of the virus (NS2, NS3, NS4, NS4A, NS4B, NS5A, and NSSB).

PCT/SE2014/051005 discloses inter alia a compound of the formula

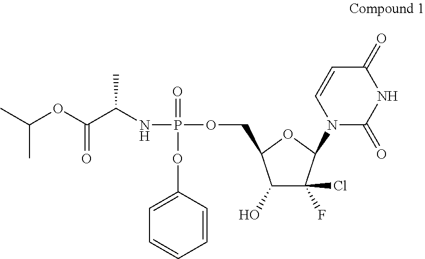 [SMILES: CC(C)OC(=O)[C@H](C)NP(=O)(OC[C@H]1O[C@@H](n2ccc(=O)[nH]c2=O)[C@@](F)(Cl)[C@@H]1O)Oc1ccccc1]

and its efficacy in the inhibition of HCV replication. It will be apparent that Compound 1 is a so-called protide, that is a phosphoramidate prodrug which releases a nucleosides monophosphate in vivo, predominantly in liver cells. The marketed HCV drug sofosbuvir

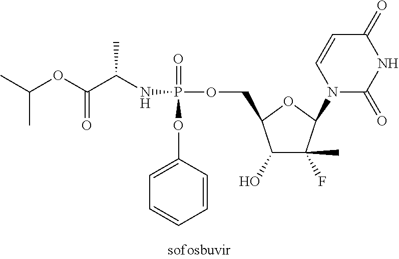 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

is a further example of such a protide. Formulation of protides for oral dosage can be difficult in view inter alia of the rigidity of the nucleoside scaffold, the contrasts in lipophilicity and polarity between different areas of the molecule and electronic effects, including the complex di-halo stereo center at the 2′ position of Compound 1. In particular, and as shown in the accompanying Examples, conventional pharmaceutically acceptable vehicles and formulations of Compound 1 tend to produce an insoluble gel when exposed to water, such as must occur during dissolution of a pharmaceutical composition in the gastric and intestinal fluid during oral administration. This gel impedes uptake of Compound 1 by the GI tract, thereby resulting in poor pharmacokinetics. Unlike sofosbuvir it has proven difficult to manufacture Compound 1 in crystalline form, ie Compound 1 is generally isolated as an amorphous material.

This invention, as comprehensively disclosed and claimed below, has been developed from the finding that Compound 1 has an extraordinary solubility in the pharmaceutically acceptable solvent ethanol. By way of reference, the published solubility of commercially available sofosbuvir varies between 25 mg/ml (Cayman Chemical, product information item no 15402 and Apex BT Catalog no. A3738) to 100 mg/ml (Selleckchem product information Catalog no S2794). In contrast, as shown in the accompanying Examples, the solubility of Compound 1 in ethanol can be orders of magnitude higher.

### BRIEF DESCRIPTION OF THE INVENTION

In accordance with a first aspect of the invention, there is provided a pharmaceutical composition comprising a compound of the formula 1:

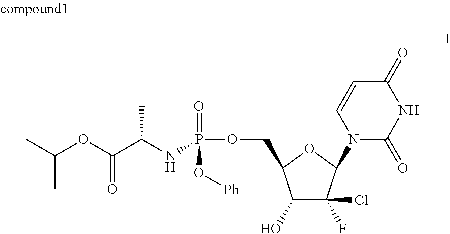 [SMILES: CC(C)OC(=O)[C@H](C)N[P@](=O)(OC[C@H]1O[C@@H](n2ccc(=O)[nH]c2=O)[C@@](F)(Cl)[C@@H]1O)Oc1ccccc1]

wherein the pharmaceutical composition further comprises ethanol.

If the other phosphorous diastereomer of compound 1 is present, it is preferred that the compound of formula I is at least 90%, preferably at least 95%, enantiomerically pure as regards the P(S) diastereomer.

The Compound 1 will typically be present in the compositions of the invention in the range 150 to 3000 mg/ml of ethanol, generally 250-2700 mg/ml, such as 250-1500 mg/ml, 250-750 mg/ml or 250-500 mg/ml, for example 300-1500 mg/ml, 300-750 mg/ml or 300-500 mg/ml.

The ability to form such concentrated solutions of Compound 1 in the pharmaceutically acceptable, and easily handled solvent ethanol provides advantages in galenic processes, such as the preparation, work-up and storage of bulk drug substance, and the preparation of combination HCV antiviral products where several antivirals of differing physicochemical ad pharmacokinetic properties must be co-formulated into common unit dosage forms, such as tablets or capsules.

For use in the various aspects of the invention the ethanol will typically be at least 95%, such as at least 98%, for example at least 99% water-free. Preferably the ethanol is 100% dehydrated ethanol.

In an embodiment of the invention, the pharmaceutical composition further comprises Solutol HS15.

In an embodiment of the above described aspect of the invention, an ethanolic solution of Compound 1, optionally in admixture with conventional pharmaceutically acceptable miscible solvents, is adsorbed to an inorganic mesoporous carrier with a specific surface area 100-1000 m²/g, such as 100-800 m²/g, thereby forming a solid carrier suitable for tableting processes or for filling in hardgel or softgel capsules. Representative inorganic mesoporous carriers include aerosil, neusilin, CaCO₃, MgCO₃ and mixtures thereof.

A further advantage of the above described solutions of Compound 1 in ethanol is that they are well adapted for the preparation of self-emulsifying drug dispersal systems.

Accordingly a second aspect of the invention provides a pharmaceutical composition in unit dosage form, comprising:
a) 200-750 mg of the compound of formula I;
b) 40-400 mg ethanol;
c) nucleation inhibitor;
d) hydrophilic surfactant with an HLB>12;
and, optionally
e) triglyceride, diglyceride, monoglyceride or mixtures thereof; and/or:
f) hydrophilic cosolvent selected from propylene glycol, polyethylene glycol, glycerol, 2-(2-ethoxyethoxy)ethanol, and mixtures thereof.

In an embodiment each unit dosage form contains 340-580 mg Compound 1, such as 400, 450 or 500 mg Compound 1.

In an embodiment, each unit dosage form comprises 50-250 mg ethanol, preferably 59-222 mg ethanol.

Pharmaceutically acceptable nucleation inhibitors useful for the invention include one or more hydrophilic polymer selected from:
homopolymers of N-vinyl lactam,
copolymers of N-vinyl lactam,
cellulose esters,
cellulose ethers,
polyalkylene oxides,
polyacrylates,
polymethacrylates,
polyacrylamides,
polyvinyl alcohols,
vinyl acetate polymers,
oligosaccharides, or
polysaccharides.

In some embodiments, the nucleation inhibitor comprises one or more hydrophilic polymers selected from homopolymer of N-vinyl pyrrolidone,
copolymer of N-vinyl pyrrolidone,
copolymer of N-vinyl pyrrolidone and vinyl acetate,
copolymer of N-vinyl pyrrolidone and vinyl propionate,
graft copolymer of polyethylene glycol/polyvinyl caprolactam/polyvinyl acetate (e.g., Soluplus),
polyvinylpyrrolidone,
methylcellulose,
ethylcellulose,
hydroxyalkylcelluloses,
hydroxypropylcellulose,
hydroxyalkylalkylcellulose,
hydroxypropylmethylcellulose,
cellulose phthalate,
cellulose succinate,
cellulose acetate phthalate,
hydroxypropylmethylcellulose phthalate,
hydroxypropylmethylcellulose succinate,
hydroxypropylmethylcellulose acetate succinate,
polyethylene oxide,
polypropylene oxide,
copolymer of ethylene oxide and propylene oxide,
methacrylic acid/ethyl acrylate copolymer,
methacrylic acid/methyl methacrylate copolymer,
butyl methacrylate/2-dimethylaminoethyl methacrylate copolymer,
poly(hydroxyalkyl acrylate),
poly(hydroxyalkyl methacrylate),
copolymer of vinyl acetate and crotonic acid,
partially hydrolyzed polyvinyl acetate,
carrageenan,
galactomannan,
xanthan gum,
or a combination thereof.

In some embodiments, the nucleation inhibitor comprises polyvinylpyrrolidone.

In some embodiments, the hydrophilic surfactant comprises:
polyoxyethylene castor oil derivates,
mono fatty acid ester of polyoxyethylene sorbitan,
polyoxyethylene alkyl ether,
polyoxyethylene alkylaryl ether,
polyethylene glycol fatty acid ester,
alkylene glycol fatty acid mono ester,
sucrose fatty acid ester, or
sorbitan fatty acid mono ester.

Representative hydrophilic surfactants include:
polyoxyethyleneglycerol triricinoleate;
polyoxyl 35 castor oil (Cremophor EL; BASF Corp.),
polyoxyethyleneglycerol oxystearate such as polyethylenglycol 40 hydrogenated castor oil (Cremophor RH 40, also known as polyoxyl 40 hydrogenated castor oil or macrogolglycerol hydroxystearate),
polyethylenglycol 60 hydrogenated castor oil (Cremophor RH 60), mono fatty acid ester of polyoxyethylene sorbitan, such as mono fatty acid ester of polyoxyethylene (20) sorbitan, e.g. polyoxyethylene (20) sorbitan monooleate (Tween 80), polyoxyethylene (20) sorbitan monostearate (Tween 60),
polyoxyethylene (20) sorbitan monopalmitate (Tween 40) or polyoxyethylene (20) sorbitan monolaurate (Tween 20),
polyoxyethylene (3) lauryl ether,
polyoxyethylene (5) cetyl ether,
polyoxyethylene (2) stearyl ether,
polyoxyethylene (5) stearyl ether,
polyoxyethylene (2) nonylphenyl ether,
polyoxyethylene (3) nonylphenyl ether,
polyoxyethylene (4) nonylphenyl ether,
polyoxyethylene (3) octylphenyl ether,
PEG-200 monolaurate,
PEG-200 dilaurate,
PEG-300 dilaurate,
PEG-400 dilaurate,
PEG-300 distearate,
PEG-300 dioleate,
propylene glycol monolaurate (e.g., lauroglycol FCC),
D-alpha-tocopheryl polyethylene glycol 1000 succinate,
sucrose monostearate,
sucrose distearate,
sucrose monolaurate,
sucrose dilaurate,
sorbitan mono laurate,
sorbitan monooleate,
sorbitan monopalmitate,
sorbitan stearate,
or a combination thereof

In certain embodiments, the surfactant comprises Solutol HS15.

In some embodiments, the hydrophilic cosolvent comprises polyethylene glycol 400.

In some embodiments, the glyceride is Capmul MCM.

Pharmaceutical compositions in accordance with the above described second aspect of the invention can adsorbed on an inorganic mesoporous carrier with a specific surface area 100-1000 m²/g such as 100-800 m²/g. Representative carriers include aerosil, neusilin, CaCO₃, MgCO₃ and mixtures thereof. Such pharmaceutical compositions are useful for including in conventional tablet formulations, or as fillers in hard shell or preferably softgel capsules.

Without wishing to be bound by theory, adsorption to a mesoporous carrier is believed to facilitate the stability of the amorphous form, by inhibiting spontaneous crystallization. Additionally, adsorption to a mesoporosu carrier is expected to decrease the tendency of Compound 1 to exhibit gelation.

Carriers will typically be microporous inorganic substances, high surface area colloidal inorganic adsorbent substances, or nanoparticle adsorbents, for example silica, silicates, magnesium trisilicate, magnesium aliminium silicate (Neusilin), microporous calcium silicate (Florite™ RE), magnesium hydroxide or talcum,

Representative carriers include silica based-materials like fumed silica nanoparticles, for example aersoli, neusilin, non-ordered mesoporous silica (Syloid) or ordered mesoporous silica based materials (OMS) like MCM series (MCM-41) or SBA series (SBA-15) or different forms of mesoporous nonsilicate oxides (MNSOs) or mesoporous CaCO₃, MgCO₃ and mixtures thereof.

Other exemplary carriers include surface-modified mesoporous silicon (thermally carbonized PSi (TCPSi) thermally oxidized Psi (TOPSi) and non-ordered mesoporous silica (Syloid AL-1 and 244).

Below is provided a list of porous adsorbent carriers having suitable properties for providing a loadable composition, e.g. tablet, according to the invention. The porous adsorbent materials may be used alone or in combination provided that the desired porosity of the composition or tablet is obtained.

To this end, it should be noted that the tablets are compressed into tablets by use of a certain compression force. However, the compression force may not be so low that the requirements with respect to hardness and friability of the tablets are compromised, i.e. these requirements ensure that the tablets are sufficiently robust.

Suitable pharmaceutically acceptable excipients that can be used to obtain tablets having a porosity of 30% v/v or more are selected from the group consisting of metal oxides, metal silicates, metal carbonates, metal phosphates, metal sulfates, derivatives. The metal is typically selected from the group consisting of sodium, potassium, magnesium, calcium, zinc, aluminium, titanium and silicon. A suitable metal oxide for use according to the invention may be selected from the group consisting of magnesium oxide, calcium oxide, zinc oxide, aluminium oxide, titanium dioxide including Tronox A-HP-328 and Tronox A-HP-100, silicon dioxides including Aerosil, Cab-O-Sil, Syloid, Aeroperl, Sunsil (silicon beads), Zeofree, Sipernat, and mixtures thereof.

In a specific embodiment, the metal oxide is a titanium dioxide or a silicon dioxide or mixtures thereof.

The silicates can be divided in the following groups:
Hydrous aluminium silicates or alkaline earths. Neusilin belongs to this group and is based on synthetic polymerisation (magnesium aluminium metasilicate).
Silicon dioxides are subdivided into porous and nonporous silicas
Nonporous colloidal silicas e.g. Aerosil (fumed silicas)
Porous silicas gels e.g. Syloid, Porasil, Lichrosorp
Others e.g. Zeopharm S170, Zeopharm 6000, Aeroperl 300 Accordingly, a loadable tablet according to the invention may contain a metal oxide that is a non-porous silicate including fumed silicas of the Aerosil type, and/or a porous silicate including e.g. Syloid, Porasil and Lichrosorp.

In other embodiments, the pharmaceutically acceptable excipient for use according to the invention is a metal silicate selected from the group consisting of sodium silicate, potassium silicate, magnesium silicate, calcium silicate including synthetic calcium silicate such as, e.g., Hubersorp, microporous calcium silicate, such as Florite, zinc silicate, aluminum silicate, sodium aluminosilicate such as, e.g., Zeolex, magnesium aluminum silicate, magnesium aluminum metasilicate, aluminium metasilicate, Neusilin SG2 and Neusilin US2 and mixtures thereof.

The aluminum silicate is a highly porous material having a typical average pore size of 30 to 80, such as 50-60 angstrom and a surface area of from 250 to 400 m²/g, such as about 300 m²/g. The composition of the present invention typically has a porosity of 30% v/v or more, which is necessary for absorption of a suitable amount of a pharmaceutically active ingredient. In further embodiments the porosity is 40% v/v or more, 50% v/v or more, 60% v/v or more, 70% v/v or more, 80% v/v or more, or 90% v/v or more. The porosity is measured on the aluminum silicate, such as Neusilin, and then it is calculated how much aluminum silicate and an optional pharmaceutically acceptable excipient, utilize of the porosity.

The porosity of the granules or tablets before loading is calculated on basis of the density of the granule or tablet pₜ and the “true density” pₛ of the ingredients. The porosity c of the granule or tablet is calculated according to the Equation 1:

$$ɛ = 1 - \frac{{ρ}_{t}}{{ρ}_{s}} \qquad Equation 1$$

The density of the granule or tablet is based on the ratio between weight and volume of the granule or tablet. The “true density” of the ingredients is based on the gas pycnometric density determined in helium using Micromeritics Accupyc 1330.

In a further embodiment the composition of the present invention the aluminum silicate is typically present in a concentration of about 20% w/w or more. It is apparent that the higher porosity desired the higher the concentration of the aluminum silicate, thus in further embodiments of the composition of the present invention the aluminum silicate is present in a concentration of about 25% w/w or more, about 30% w/w or more, about 35% w/w or more, about 40% w/w or more, about 45% w/w or more, about 50 w/w or more, about 60% w/w or more, about 70% or more, about 80% or more, about 90% or more, about 95% or more, or about 98% or more, in the unloaded composition.

The aluminum silicate typically, has an average pore size of 30 to 80, such as 50-60 angstrom and a surface area of from 250 to 400 m²/g, such as about 300 m²/g. In an embodiment the aluminum silicate is selected from magnesium aluminum metasilicate, magnesium aluminum silicate, and aluminium metasilicate, and mixtures thereof. Typical examples of aluminum silicates are Neusilin SG2, and Neusilin US2, and mixtures thereof, in particular Al₂.MgO.ySiO₂. xH₂O, wherein y is from 1.5-2, and x is 1-10, preferred is magnesium aluminum metasilicate, e.g. Al₂O₃.Mg0.2SiO₂.5H₂O.

As mentioned above a suitable pharmaceutically acceptable excipient may be a metal carbonate such as a carbonate selected from the group consisting of sodium carbonate, sodium hydrogen carbonate, potassium carbonate, potassium hydrogen carbonate, calcium carbonate, magnesium carbonate, zinc carbonate and aluminum carbonate, and mixtures thereof.

Other metal salt suitable for use according to the invention are metal phosphates selected from the group consisting of sodium phosphate, disodium hydrogen phosphate, sodium dihydrogen phosphate, potassium phosphate, dipotassium hydrogen phosphate, potassium dihydrogen phosphate, calcium phosphate, magnesium phosphate, zinc phosphate and aluminum phosphate. More specifically, the pharmaceutically acceptable excipient may be a calcium phosphate selected from the group consisting of dibasic anhydrous calcium phosphate, dibasic dihydrate calcium phosphate, and tribasic calcium phosphate.

The dibasic anhydrous calcium phosphate is typically selected from the group consisting of A-Tab, calcium monohydrogen phosphate, calcium orthophosphate, Di-Cafos A N, dicalcium orthophosphate, E341, Anhydrous Emcompress, Fujicalin, phosphoric acid calcium salt (1:1), and secondary calcium phosphate, and mixtures thereof. The dibasic dihydrate calcium phosphate may be selected from the group consisting of Cafos, calcium hydrogen orthophosphate dihydrate, calciummonohydrogen phosphate dihydrate, Calipharm, Calstar, Di-Cafos, dicalcium orthophosphate, DI-TAB, Emcompress, phosphoric acid calcium salt (1:1) dihydrate, secondary calcium phosphate, Fujiclin S G.

Examples of tribasic calcium phosphates are e.g. hydroxyapatite, phosphoric acid calcium salt (2:3), precipitated calcium phosphate, tertiary calcium phosphate, Tri-Cafos, tricalcium diorthophosphate, tricalcium orthophosphate, tricalcium phosphate, TRI-CAL, WG, TRI-TAB.

Other suitable metal salts are metal sulfates such as, e.g, sodium sulfate, sodium hydrogen sulfate, potassium sulfate, potassium hydrogen sulfate, calcium sulfate, magnesium sulfate, zinc sulfate and/or aluminum sulfate.

Examples of suitable calcium sulfates are e.g. calcium sulfate anhydrous including anhydrite, anhydrous gypsum, anhydrous sulfate of lime, Destab, Drierte, E516, karstenite, muriacite, and Snow White or calcium sulfate dihydrate including alabaster, Cal-Tab, Compactrol, Destab, E516, gypsum, light spar, mineral white, native calcium sulfate, precipitated calcium sulfate, satinite, satin spar, selenite, terra alba and USG Terra Alba.

Any one of the above porous adsorbent materials are intended to be embodiments of the invention as long as they alone or in mixture provides a suitable porosity as described above. The below specified embodiments are not to be construed as limiting the invention in any way but are merely to highlight certain preferred embodiments.

In a further embodiment, the porous abdsorbent material is selected from porous silicon dioxide, such as sodium silicate, potassium silicate, magnesium silicate, calcium silicate, including synthetic calcium silicate, microporous calcium silicate, zinc silicate, aluminum silicate, sodium aluminosilicate, hydrous aluminium silicates or alkaline earths, magnesium aluminum metasilicate, magnesium aluminum silicate, aluminium metasilicate, nonporous colloidal silicas, porous silicas gels, precipitated silicate, and mixtures thereof. In a further embodiment the porous adsorbent material is selected from metal carbonates and metal phosphates. Typically, the porous adsorbent material is selected from magnesium aluminum metasilicate, precipitated silicate, and microporous calcium silicate.

Although the invention has thus far been described with reference to the extraordinary solubility of Compound 1 in ethanol, it should be appreciated that certain SEDDS formulations can be applied to Compound 1, even without necessarily including an ethanolic solution. Accordingly a third aspect of the invention provides a pharmaceutical composition comprising a compound of the formula I:

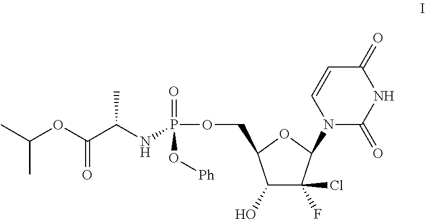 [SMILES: CC(C)OC(=O)[C@H](C)N[P@](=O)(OC[C@H]1O[C@@H](n2ccc(=O)[nH]c2=O)[C@@](F)(Cl)[C@@H]1O)Oc1ccccc1]

in a pharmaceutically acceptable vehicle comprising w:w
a) 0-80% trigyceride, diglyceride, monoglyceride or mixtures thereof,
b) 0-50% hydrophilic cosolvent selected from ethanol, propylene glycol, polyethylene glycol, glycerol, 2-(2-ethoxyethoxy)ethanol, and mixtures thereof;
c) nucleation inhibitor;
d) hydrophilic surfactant with an HLB>12;
   wherein the % w:w ratio of a:b:c:d is selected from the table below and totals 100:

| SEDDS form | a) | b) | c) | d) |
|---|---|---|---|---|
| Pouton | 40-80 | 0-40 | 0.01-10 | 20-40 |
| Type IIIA | | | | |
| Pouton | <20 | 20-50 | 0.01-10 | 20-50 |
| Type IIIB | | | | |
| Pouton | — | 0-50 | 0-10 | 30-95 |
| Type IV | | | | |

As with the earlier described aspects, the compound of formula I is generally at least 90%, preferably at least 95%, enantiomerically pure as regards the P(S) diastereomer.

The nucleation inhibitor, hydrophilic surfactant and triglyceride are all as defined above. The hydrophilic cosolvent preferably includes ethanol, especially when the pharmaceutical composition is a Pouton type IV SEDDS.

### EXAMPLES

Various embodiments, preparative examples and comparative examples are disclosed in further detail in the following examples, which are not in any way intended to limit the scope of the claims.

### Example 1. Preparation of Compound 1

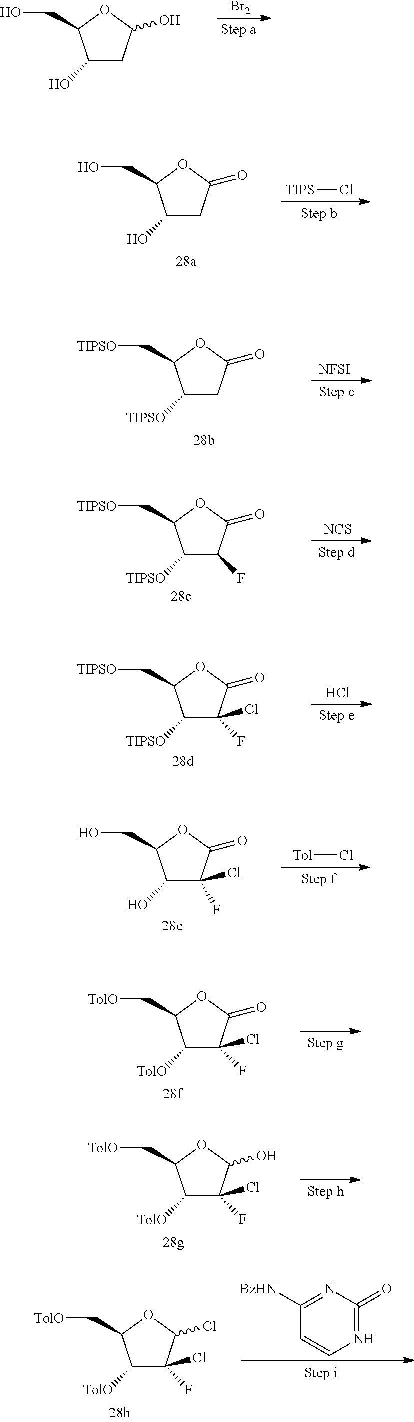

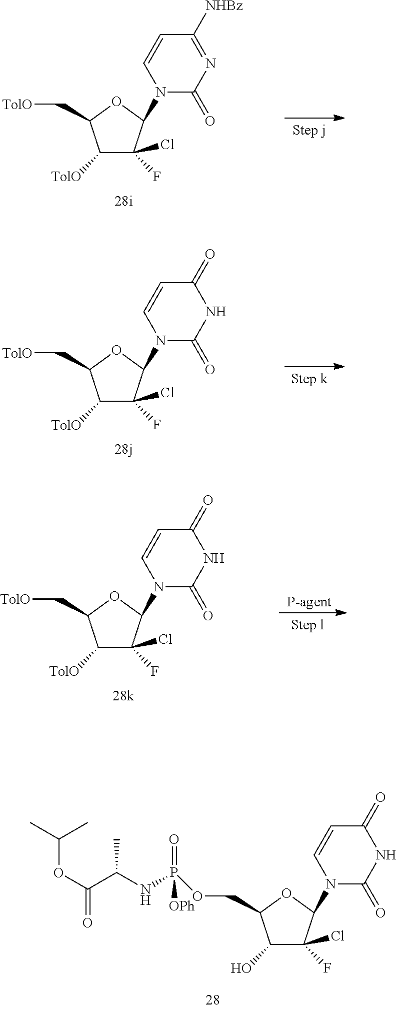

### Step a) (4S,5R)-4-Hydroxy-5-(hydroxymethyl)dihydrofuran-2(3H)-one (28a)

Deoxy-D-ribose (400.0 g, 2.98 mol) was dissolved in water (1.6 kg) under nitrogen and the solution cooled to 3-7° C. Bromine (800 g, 10.0 mol, 3.36 eq.) was added at 3-7° C. while stirring over a period of approximately 2 hours and the stirring was continued at 3-7° C. for approximately 1 hour. The reaction mixture was gently warmed to 20-25° C. and then stirred for approximately 20 hours.

The reaction mixture was cooled to −5 to −7° C. and a solution of sodium hydroxide (27.65%, 720 g, 1.67 eq.) was added while keeping the reaction temperature at −3 to −7° C. The temperature was then adjusted to 0-5° C. and aqueous sodium hydroxide (9%, 470 g, 1.06 mol, 0.35 eq. was added at 0-5° C. to obtain a final pH=1.40.

The water was distilled off at reduced pressure using a scrubber (cooled, 14% sodium hydroxide, 0.9 L), finally at p<5 mbar and 50° C. In order to remove residual water from the product, 2-propanol was added portion wise to the residue followed by azeotropic distillation at reduced pressure. The final water content was determined by KF titration to be less than 1%. 2-Propanol (400 mL) was added to the residue and the mixture followed by filtration. The filter cake was washed with 2-propanol (1 L). The solvent was distilled off at reduced pressure. Toluene (400 mL) was added and distillation was resumed in order to remove residual 2-propanol and possibly more water. A residue of 474.6 g (120% yield) was obtained.

### Step b) (4S,5R)-4-((Triisopropylsilyl)oxy)-5-(((triisopropylsilyl)oxy)methyl)dihydrofuran-2(3H)-one (28b)

Compound 28a (470.9 g, 2.97 mol) was dissolved in DMF (1.2 L) and cooled to 10-15° C. Imidazole (707.0 g, 10.4 mol, 3.5 eq.) was added and the temperature of the mixture was adjusted to 3-7° C. TIPS-CI (1145 g, 5.94 mol, 2.0 eq.) was added with cooling to 3-7° C. over a period of 2 hours. The reaction mixture was stirred at 3-7° C. for another ½ h, then gently warmed to 20-25° C. and stirred for 20 h. The progress of the reaction was monitored as follows: A sample of the reaction mixture was diluted 10 times with dry DMF, N;O-bis(trimethylsilyl)trifluoroacetamide (0.25 mL) was added to 0.5 mL of the sample in DMF and analyzed by GC. If the reaction was not complete the necessary amount of TIPS-CI was calculated and added and the stirring continued for another 20 hours.

When the reaction was completed, methanol (50 mL) was added and the mixture was stirred for ½-1 hour at 20-25° C. Water (1.2 kg) was added and the temperature of the mixture was adjusted to 15-25° C. pH was adjusted to pH 2.0-2.5 by careful addition of 36% hydrochloric acid (491 g, 4.7 mol). Toluene (0.9 kg) was added and the phases were separated. The organic phase was washed twice with 5% aqueous sodium chloride (1 kg). the aqueous phases were washed with toluene (0.9 kg). The organic phases were combined and dried with sodium sulfate (150 g) for minimum 1 hour. The suspension was filtered on a column prepared from silica Gel 60 (210 g) and toluene and the column was washed with toluene (1.1 kg). The combined filtrate was concentrated to dryness at reduced pressure at 50° C. which gave the title compound (1338 g, 84.4% from crude 2a). Purity (GC): 93.9%.

### Step c) (3S,4R,5R)-3-fluoro-4-((triisopropylsilyl)oxy)-5-(((triisopropylsilyl)oxy)methyl)-dihydrofuran-2(3H)-one (28c)

Compound 28b (450.0 g, 1.01 mol,) and NFSI (348.0 g, 1.10 mol) were dissolved in Me-THF (2.2 L) under argon. The solution was cooled to below −75° C. and lithium bis(trimethylsilyl)amide (20.2% in THF, 1.190 kg, 1.42 eq.) was added over a period of 3-4 hours. The progress of the reaction was monitored by GC, and when deemed completed, methylsulfide (6 g, 0.1 mol) was added to quench residual NFSI and the stirring continued for another 20-30 minutes. The reaction mixture was transferred into aqueous 12.5% ammonium chloride (1.7 kg) and the mixture was warmed to room temperature. The aqueous layer (Aq. 1) was separated and the organic phase was washed with purified water (1 L). The aqueous wash (Aq. 2) was separated and the organic phase was secured. Aq.1 was washed with heptanes (0.6 kg). The aqueous phase was separated and then discarded. Aq. 2 was added to the organic phase and the mixture was stirred for 1 minute. The aqueous phase was separated and discarded. The two organic phases were combined and concentrated at reduced pressure at 50° C. Heptanes (0.7 kg) was added to the residue and the resulting suspension was filtered. The filter cake was washed with heptanes (0.2 kg), the combined filtrate was concentrated at reduced pressure at 50° C., which gave 506 g crude product. The crude product was dissolved in a mixture of heptanes and toluene (0.5 L, 3:1) and purified by column chromatography on silica gel (silica gel 60, 2.5 kg and heptanes/toluene 3:1 v/v). The column was eluted with heptanes/toluene (3:1, 5.0 L), heptanes/toluene (2:1, 2.5 L), heptanes/toluene (3:1, 2.5 L) and toluene (7.5 L). Fractions of ˜1 L were collected and fractions holding pure compound 2c were combined and concentrated and fractions holding mixtures of compound 2c and di-fluoro compound were combined and re-purified.

The above procedure was repeated twice, starting with 450 g and 525 g of compound 2b. Total yield of the title compound was 877.1 g (59.2%)+104.1 g (7.0%) from reworked material. Purity (GC): 92.4%.

### Step d) (3S,4R,5R)-3-Chloro-3-fluoro-4-((triisopropylsilyl)oxy)-5-(((triisopropylsilyl)oxy)methyl)-dihydrofuran-2(3H)-one (28d)

Compound 28c (400.0 g, 0.86 mol) and NCS (138.0 g, 1.04 mol, 1.2 eq.) were stirred in THF (2.0 L) under argon at −20° C. The suspension was cooled to below −70° C. and then lithium bis(trimethylsilyl)amide (20.2% in THF, 1.150 kg, 1.6 eq.) was added over a period of 1-1.5 hours. The reaction was monitored by GC and when deemed completed, the mixture was transferred into a 12.5% aqueous solution of ammonium chloride (1.5 kg). The mixture was warmed to room temperature. The stirring was stopped and the aqueous layer was separated, washed with heptanes (0.8 L) and then discarded.

The mother organic phase was concentrated to dryness at reduced pressure at 55° C. and then added to the heptane wash. The thus combined organic phases were washed with 5% aqueous sodium chloride. The phases were separated and the aqueous phase washed with heptanes (0.2 L), then discarded. The organic phase was concentrated at reduced pressure which gave 440 g of crude product.

The procedure was repeated starting with 426.5 g of compound 2c which gave 473 g of crude product.

The combined crude products were dissolved in a mixture of heptanes and toluene (1.0 L, 2:1) and purified on a silica gel column prepared from silica gel 60 (2.25 kg) and heptanes/toluene 2:1 v/v. The column was eluted with: heptanes/toluene (2:1, 15 L). Fractions of −1 L were collected and pure fractions of compound 2d were combined and concentrated at reduced pressure which gave the title compound (667.3 g, 75.1%).

### Step e) (3S,4R,5R)-3-Chloro-3-fluoro-4-hydroxy-5-(hydroxymethyl)dihydrofuran-2(3H)-one (28e)

Compound 28d (613.0 g, 1.11 mol) was added to a 3 L glass reactor filled with nitrogen and methanol (1.2 L) and. To the stirred emulsion was added 37% hydrochloric acid (368.0 g, 3.73 mol, 3.4 eq.) and the mixture was heated to gentle reflux (73° C.). The mixture was kept at reflux for 20 hours then cooled to 15-20° C. and extracted with heptanes (4×600 mL). The residual methanolic solution was concentrated to dryness at reduced pressure using a water bath of 80-90° C., finally at p<35 mbar. Dioxane (600 mL) was added and distilled again as above, which gave the title compound (200.7 g, 98%).

### Step f (2R,3R,4S)-4-chloro-4-fluoro-2-(((4-methylbenzoyl)oxy)methyl)-5-oxotetrahydrofuran-3-yl 4-methylbenzoate (28f)

A solution of compound 28e (200.7 g, 1.11 mol) in dioxane (1.4 L) in a 3 L glass reactor filled with nitrogen and equipped with mechanical stirring, thermometer and an addition funnel was heated to 40 to 45° C. on a water bath. p-Toluoyl chloride (360.5 g, 2.33 mol, 2.1 eq.) was added whereafter triethylamine (258.3 g, 2.55 mol, 2.3 eq.) was added during 35 minutes so as to keep the reaction temperature below 70° C. The resulting suspension was then stirred at 65° C. for 2 hours, then cooled to 15° C. and filtered. The 800 mL filter cake was washed with dioxane (800 mL, 15° C.), leaving a white filter cake which was discarded. The filtrate was concentrated at reduced pressure, finally at 35 mbar using a water bath of 65° C. 2-Propanol (1.50 L) was added to the residual oil (510 g) so as to keep the temperature of the solution at 40-45° C. The solution was seeded and carefully allowed to cool to room temperature. During the cooling process samples of 0.25 mL were taken and mixed with 0.25 mL of water for pH measurements. Triethylamine (15 g) was added until pH 2.5-3.5 was obtained. Once room temperature was reached (one hour), the crystal suspension was cooled to 10±1° C. and kept at this temperature for 15 hours. The title product was isolated by filtration, washed with 2-propanol (600 mL, 5-10° C.) and then dried at 30-50° C. in an air vented oven. Yield: 374.2 g, 80%. Purity (HPLC): 99.4%. Melting point: 88.0-89.5° C. (1° C./min) crystal form change and then melts at 97-98° C.

### Step g) (2R,3R,4S)-4-Chloro-4-fluoro-5-hydroxy-2-(((4-methylbenzoyl)oxy)methyl)-tetrahydrofuran-3-yl 4-methylbenzoate (28g)

A 3 L reaction flask set up with mechanical stirrer, thermometer and an addition funnel was filled with nitrogen. The flask was charged with ethyl acetate (1000 g) and cooled to 10° C. Lithium tri-tert-butoxyaluminium hydride (30% solution in THF, 35 g, 0.05 eq.) was added. Stirring at 10° C. was continued for 5-10 minutes and then compound 28f (370.0 g, 0.88 mol) was added. Further lithium tri-tert-butoxyaluminium hydride (30% solution in THF, 933.8 g, 1.10 mol, 1.25 eq.) was added over a period of 70 minutes while keeping the reaction temperature at 10° C. The reaction was quenched by pouring the reaction mixture onto a quench mixture (1.45 kg (10% NaCl-10% NH₄Cl in 3M HCl)) keeping the temperature at 10-15° C. The resulting suspension was warmed to 20-25° C. The aqueous was separated and discarded and the organic phase was washed with acidic water (1.0 L+10 mL of 3M HCl) followed by a wash with 25% sodium chloride (250 mL). The organic phase was concentrated to dryness, finally at p<35 mbar and 45° C. The residue was re-dissolved in toluene (0.45 kg) and the solution was again concentrated, at p<35 mbar and 45° C., which gave the title compound as an oil containing a little solid sodium chloride (412.6 g, 111%). Purity (HPLC) 97.5%.

### Step h) (2R,3R,4S)-4,5-dichloro-4-fluoro-2-(((4-methylbenzoyl)oxy)methyl)tetrahydrofuran-3-yl 4-methylbenzoate (28h)

A 2000 mL reaction flask set up for mechanical stirring, temperature measurement and condenser was filled with nitrogen and charged with toluene (740 mL), compound 28g (411.5 g, 0.88 mol) and thionyl chloride (174.0 g, 1.46 mol, 1.66 equivalents). The reaction flask was placed on a water bath, pre-heated to 50° C. and DMF (0.50 mL) was added. The top of the condenser was connected a cooled scrubber (700 g of 27.65% sodium hydroxide) and a steady flow of nitrogen was applied. The reaction started shortly after the DMF was added and it was followed by HPLC. After approximately three hours, the gas evolution has decreased and the temperature was increased to 60-65° C. Heating at 60−65° C. was continued for further 4.5 hours after which time the sulfite esters had vanished. The solvent and residual thionyl chloride was distilled off (500 mL) at reduced pressure using a water bath of 60-65° C. Toluene (650 mL) was added to the residual oil and the mixture was cooled to 5° C. Water (650 mL) was added and the pH was adjusted to 2.0-3.0 by addition of 3M sodium hydroxide (40 mL) at a temperature below 10° C. The temperature was adjusted to 20-22° C. and the aqueous phase was separated. The organic phase was washed with 25% sodium chloride (250 mL). The aqueous phases were back washed with toluene (250 mL). The combined organic phase was dried with magnesium sulfate (25 g) and filtered. Evaporation of the solvent (finally at p<35 mbar and 60° C.) provided the title compound as a light brown oil (378.5 g, 97% yield). Chlorobenzene (200 g) was added to the residue and the mixture was concentrated using the above conditions. The residue was again dissolved in chlorobenzene (200.0 g) and the mixture concentrated.

### Step i) (2R,3R,4S,5R)-5-(4-Benzamido-2-oxo-3,4-dihydropyrimidin-1(2H)-yl)-4-chloro-4-fluoro-2-(((4-methylbenzoyl)oxy)methyl)tetrahydrofuran-3-yl 4-methylbenzoate (28i)

A 500 mL round bottom flask was charged with N-benzoylcytosine (36.6 g, 170 mmol, 1.5 eq.), chlorobenzene (165 g, 150 mL) and ammonium sulfate (0.45 g, 3.4 mmol, 0.03 eq.), to this suspension was added HMDS (29.3 g, 181.3 mmol, 1.6 eq.). The suspension was heated to reflux. When the reaction mixture became a clear solution, it was refluxed for additional 1 h and then concentrated by distillation in vacuo at 60° C. (distillate: 150 mL). Chlorobenzene (125 mL) was added to the residue.

Residual toluene in Compound 28h (50 g, 113.3 mmol) was removed by distillation in vacuo from chlorobenzene. The residue from this co-evaporation was dissolved in 1,2-dichloroethane (200 mL), and this solution was charged to the solution of silylated nucleoside in chlorobenzene. Tin(IV)chloride (59.0 g, 226.6 mmol, 2 eq.) was added and the mixture was heated to reflux under nitrogen. The reaction mixture was stirred at reflux for 65 h. The reaction mixture was cooled to 5° C., and ethyl acetate (99.8 g, 10 eq.) was added while keeping the temperature at 10-12° C. Total weight of mixture: 601.7 g. A quarter of this mixture (150.4 g, in theory 28.3 mmol) was charged to a 250 mL 3 necked round bottom flask, cooled to 5° C., and dichloromethane (147.5 g, 4×vol. of EtOAc) was added together with Celite (6.25 g). A warm (approx. 60° C.) 50% NaOH solution (17.6 g, 7.76 eq.) was added to the mixture in such a rate that the temperature was kept at 5-12° C. The mixture was stirred for 20 min at 10° C., then the temperature was adjusted to 25° C. and the mixture was stirred at this temperature for 30 min. The suspension was filtered on a pad of Celite (12.5 g) and the filter cake was washed with dichloromethane (190 mL). The combined filtrate and washings were concentrated to dryness by distillation in vacuo at 60° C.

Dichloromethane (86 mL) was added to the residue then toluene (62 mL). The content of dichloromethane was removed by distillation in vacuo at 50° C. The resulting suspension was stirred at room temperature for 17 h whereafter the crude title compound was isolated by filtration. The filter cake was washed with toluene (25 mL) and the wet product was dried in an air ventilated dryer at 40° C., which gave title compound as a solid (5.56 g, 31.7%).

### Step j) (2R,3R,4S,5R)-4-Chloro-5-(2,4-dioxo-3,4-dihydropyrimidin-1(2H)-yl)-4-fluoro-2-(((4-methylbenzoyl)oxy)methyl)tetrahydrofuran-3-yl 4-methylbenzoate (28j)

Compound 28i (15.2 g 24.5 mmol) was suspended in 65% AcOH/water (152 mL, v/v), and the suspension was heated to reflux for 20 h. The reaction mixture was allowed to cool to room temperature, then water (53 mL) was added and the mixture was stirred at room temperature for 1.5 h. The suspension was filtrated and the filter cake washed with water (2×25 mL). The wet filter cake was dried in an air ventilated dryer at 40° C. for 20 h, which gave the title compound as a solid (10.8 g, 85%).

### Step k) 1-((2R,3S,4R,5R)-3-Chloro-3-fluoro-4-hydroxy-5-(hydroxymethyl)tetrahydrofuran-2-yl)pyrimidine-2,4(1H,3H)-dione (28k)

Compound 28j (8.0 g, 15.5 mmol) was suspended in MeOH (80 mL), n-propylamine (9.1 g, 154.8 mmol, 10 eq.) was added and the mixture was heated to 30° C. and stirred at this temperature for 24 h. The solvents were removed by distillation in vacuo at 40° C. The residue was taken up in water (20 mL), the aqueous phase was washed with DCM (3×40 mL) and the combined organic phases were washed with water (5 mL). The two aqueous phases were combined, and pH adjusted to 1.0 with 3 M HCl (approx. 7 mL). The acidic aqueous phase was extracted with Me-THF (4×40 mL), and the combined organic phases were concentrated to dryness by distillation in vacuo at 40° C. Isopropyl acetate (80 mL) was added to the residue, and the turbid mixture was concentrated in vacuo at 60° C. Isopropyl acetate (40 mL) was added and the distillation in vacuo was continued. Isopropyl acetate (10 mL) was added to the resulting thick suspension. The suspension was cooled to room temperature and stirred for 30 min. Crude title compound was collected by filtration, and the filter cake was washed with isopropyl acetate (2×4 mL). The afforded crude was dissolved in Me-THF (35 mL), isopropyl acetate (70 mL) was added and the mixture was concentrated by distillation in vacuo at 60° C. (distillate: 70 mL). Additional isopropyl acetate (30 mL) was added, and the distillation was continued (distillate: 30 mL). The suspension was cooled to room temperature, stirred at for 45 min and then filtered. The filter cake was washed with isopropyl acetate (2×4 mL) then dried in vacuo at room temperature. The title compound was isolated in 70% yield (3.0 g) as an amorphous solid. Purity (HPLC) 98.5%.

### Step l) (S)-Isopropyl 2-(((S)-(((2R,3R,4S,5R)-4-chloro-5-(2,4-dioxo-3,4-dihydropyrimidin-1(2H)-yl)-4-fluoro-3-hydroxytetrahydrofuran-2-yl)methoxy)(phenoxy)phosphoryl)amino)propanoate (28)

THF (0.07% water, 12 mL) was added to Compound 28k (500 mg, 1.78 mmol) and the solution was cooled to −10° C. under nitrogen. Tert-butylmagnesium chloride, 20% wt in THF (2.20 g, 3.74 mmol, 2.1 eq.) was added by syringe over 20 min at −10° C. The syringe was rinsed with 5004 THF and the rinse was added to the reaction mixture. The formed suspension was stirred at −10° C. for 40 min. A solution of (S)-isopropyl-2(((S)(perfluorophenoxy)(phenoxy)phosphoryl)-amino) propanoate (1.01 g, 2.23 mmol, 1.25 eq.) in THF (10 mL) and DMPU (2.0 mL, 16.9 mmol, 9.5 eq.) was added with a syringe at −10° C. over a period of 87 min whereafter the reaction mixture was stirred at −10° C. for 22 h. The reaction was quenched by addition of 1 M HCl (4.6 mL, 2.6 eq.) while keeping the temperature below 5° C. Toluene (20 mL) was added and the mixture was heated to 25° C. and stirred at this temperature for 5 min. The phases were separated and the aqueous phase was extracted with toluene/THF (1:1, 10 mL). The organic phases were washed with 1 M HCl (2×10 mL) and 5% Na₂CO₃ (2×10 mL). The combined basic aqueous phases were extracted with toluene (1×10 mL) and toluene/THF (1:1, 2×10 mL) and the combined organic phases were washed with 25% NaCl (15 mL). All organic phases were then combined and the solvents removed by distillation in vacuo at 60° C. 2-Propanol (20 mL) and n-heptane (30 mL) was added to the residue and the suspension was cooled to 5° C. overnight. The suspension was filtered and the filtrate was concentrated by distillation in vacuo at 50° C. The residue was dried on a pump for 3 h which gave the title compound as a foam (874 mg, 89%). Purity (HPLC) of crude 91.8%.

NMR spectra obtained for compound 28 were in agreement with published spectral data:

¹H NMR (500 MHz, DMSO) δ 1.15 (d, 6H), 1.23 (d, 3H), 3.80 (tq, 1H), 4.04 (m, 1H), 4.31 (m, 3H), 4.86 (hept, 1H), 5.63 (dd, 1H), 6.09 (dd, 1H), 6.24 (d, 1H), 6.66 (d, 1H), 7.21 (m, 3H), 7.38 (m, 2H), 7.58 (d, 1H), 11.63 (m, 1H).

¹³C NMR (126 MHz, DMSO) δ 19.64 (d), 21.26, 21.30, 49.67, 64.32, 67.89, 74.42 (d), 78.81, 87.60 (m), 102.27, 113.96 (d), 119.96 (d), 124.52, 129.56, 139.91, 150.01, 150.53 (d), 162.52, 172.45 (d).

³¹P NMR (162 MHz, DMSO) δ 3.76.

¹⁹F NMR (376 MHz, DMSO) δ−119.05.

### Example 1A Preparation of Chiral Phosphoramidate Reagent

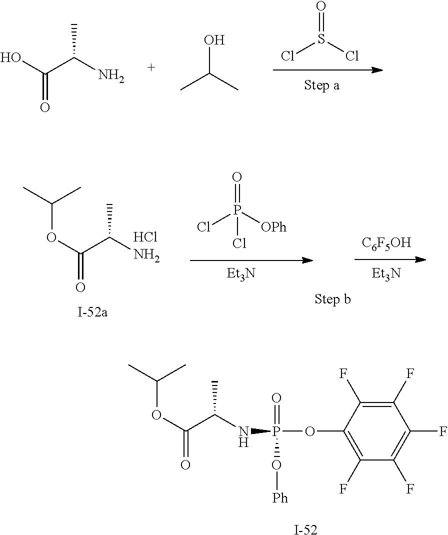

### Step a) L-Alanine isopropylester hydrochloride (I-52a)

Thionylchloride (80.2 g, 0.674 mol, 1.5 eq) was added with cooling to 2-propanol (400 mL) at −7 to 0° C. over a period of 30 minutes, followed by addition of L-alanine (40.0 g, 0.449 mol) at 0° C. A flow indicator and a scrubber with a mixture of 27.65% sodium hydroxide (228 g) and water (225 g) were attached to the outlet. The reaction mixture was stirred at 67° C. for two hours, then at 70° C. for one hour and at 20-25° C. overnight. The reaction mixture was distilled at 47-50° C. under reduced pressure (250-50 mBar) from a 60° C. bath. When the distillation became very slow, toluene (100 mL) was added to the residual oil, and the distillation at 48-51° C. under reduced pressure (150-50 mBar) from a 60° C. bath was continued until it became very slow. t-butylmethylether (tBME)(400 mL) was added to the residual oil, and the two-phase system ws seeded under efficient stirring at 34-35° C. When crystallization was observed the mixture was cooled to 23° C. over a period of one hour, and the precipitate isolated by filtration. The filter cake was washed with tBME (100 mL) and dried to constant weight under reduced pressure without heating, which gave the title compound (67.7 g, 90%) as white solids.

### Step b) (S)-Isopropyl 2-(((S)-(perfluorophenoxy)(phenoxy)phosphoryl)amino)propanoate (I-52)

Phenyl dichlorophosphate (62.88 g, 0.298 mol, 1.0 eq) was added under nitrogen to a solution of L-alanine isopropylester hydrochloride (50.0 g, 0.298 mol) in DCM (310 mL) at 0° C.—the addition was completed by wash with DCM (39 mL). The mixture was cooled and triethylamine (63.35 g, 0.626 mol, 2.1 eq) was added over a period of 70 minutes with cooling keeping the temperature not higher than −14° C., the addition was completed by wash with DCM (39 mL). The mixture was stirred for one hour at −15 to −20° C., then heated to −8° C. and a solution of pentafluorophenol (60.38 g, 0.328 mol, 1.1 eq) and triethylamine (33.19 g, 0.328 mol, 1.1 eq) in DCM (78 mL) was added over a period of 42 minutes with cooling keeping the temperature not higher than 0° C.—the addition was completed by wash with DCM (39 mL). The mixture was stirred for one hour at 0° C. and then over night at +5° C. The formed precipitate was removed by filtration, and the filter cake washed with DCM (95 mL). The combined filtrates were washed at 5° C. with water (2×190 mL). The organic phase was distilled at 32-38° C. at reduced pressure (650-600 mBar), and distillation was continued until a residual volume of approx. 170 mL partly crystallized mass was obtained. Ethyl acetate (385 mL) was added, and the resulting clear solution was distilled at 43-45° C. under reduced pressure (300-250 mBar). Distillation was continued until a residual volume of approx. 345 mL was obtained. The clear solution was cooled to 36° C., and crystallization is induced by addition of seed crystals of (S)-isopropyl 2-(((S)(perfluorophenoxy)(phenoxy) phosphoryl)amino) propanoate (20 mg) prepared as described in J. Org. Chem., 2011, 76, 8311-8319. The mixture was cooled to 27° C. over a period of one hour, then n-heptane (770 mL) was added over a period of 47 minutes, and the mixture was stirred for an additional period of 37 minutes. Triethylamine (6.03 g, 0.2 eq) was added, and the mixture was stirred at 23-25° C. overnight. The precipitate was isolated by filtration. The filter cake was washed with ethyl acetate:n-heptane (1:9, 80 mL) and dried to constant under reduced pressure (below 0.1 mBar) without heating, which gave the title compound (75.64 g, 56%) as a white crystalline material.

¹H NMR (CDCl₃, 300 MHz) δ 7.38-7.32 (m, 2H), 7.27-7.24 (m, 2H), 7.23-7.19 (m, 1H), 5.10-4.98 (m, 1H), 4.20-4.08 (m, 1H), 4.03-3.96 (m, 1H), 1.46 (dd, 7.2, 0.6 Hz, 3H), 1.26-1.23 (2×d, 6H);

¹³CNMR (CDCl₃, 100 MHz) δ 172.7 (d, J=8.8 Hz), 150.4 (d, J=7.1 Hz), 143.4-143.0 (m), 141.0-140.2 (m), 140.0-139.8 (m), 137.6-137.2 (m), 136.8-136.2 (m), 130.0 (d, J=0.82 Hz), 125.8 (d, J=1.4 Hz), 120.3 (d, J=5.0 Hz), 69.8, 50.6, (d, J=1.9 Hz), 21.8 (d, J=1.9 Hz), 21.2 (d, J=4.4 Hz);

The crystallization properties and NMR spectral data of the title compound were in agreement with published data (J. Org. Chem., 2011, 76, 8311-8319), thus confirming the S stereochemistry of the phosphorus atom of the title compound.

### Example 2. Replicon Assay

The compositions of the invention may be examined for activity in the inhibition of HCV RNA replication in a cellular assay aimed at identifying compounds that inhibit a HCV functional cellular replicating cell line, also known as HCV replicons. A suitable cellular assay is based on a bicistronic expression construct, as described by Lohmann et al. (1999), Science vol. 285 pp. 110-113 with modifications described by Krieger et al. (2001), Journal of Virology 75: 4614-4624, in a multi-target screening strategy.

The assay utilizes the stably transfected cell line Huh-7 luc/neo (hereafter referred to as Huh-Luc). This cell line harbors an RNA encoding a bicistronic expression construct comprising the wild type NS3-NS5B regions of HCV type 1 b translated from an Internal Ribosome Entry Site (IRES) from encephalomyocarditis virus (EMCV), preceded by a reporter portion (FfL-luciferase), and a selectable marker portion (neo^{R}, neomycine phosphotransferase). The construct is bordered by 5′ and 3′ NTRs (non-translated regions) from HCV type 1 b. Continued culture of the replicon cells in the presence of G418 (neo^{R}) is dependent on the replication of the HCV RNA. The stably transfected replicon cells that express HCV RNA, which replicates autonomously and to high levels, encoding inter alia luciferase, are used for screening the antiviral compounds.

The replicon cells are plated in 384 well plates in the presence of the test and control compounds which are added in various concentrations. Following an incubation of three days, HCV replication is measured by assaying luciferase activity (using standard luciferase assay substrates and reagents and a Perkin Elmer ViewLux™ ultraHTS microplate imager). Replicon cells in the control cultures have high luciferase expression in the absence of any inhibitor. The inhibitory activity of a compound on luciferase activity is monitored on the Huh-Luc cells, enabling a dose-response curve for each test compound. EC₅₀ values are then calculated, which value represents the amount of the compound required to decrease the level of detected luciferase activity by 50%, or more specifically, the ability of the genetically linked HCV replicon RNA to replicate.

Compound 1 shows an EC₅₀ value of 0.055 uM (n>10), with a cell toxicity in the Huh-Luc cell line being in excess of 50 μM.

### Example 3—Comparative Example—Dissolution of Compound 1 in Capsules

Dissolution test. 350 mg of Compound 1 was filled into a hard gelatin capsule. A basket dissolution test was performed according to USP method 711, at 100 rpm at 37° C., 900 g media, 5 ml sampling after 20, 45, 90 minutes. Samples were analysed by RP-UPLC. The capsule collapsed into a water insoluble lump that did not dissolve or change much during 90 min. The lump was very sticky when wet but dried to a hard lump in air. It was observed that the lump was surprisingly soluble in 70% EtOH.

Quantitative results (Mean % Compound 1 released, n = 2)

| Media | 20 min | 45 min | 90 min |
|---|---|---|---|
| 0.1M HCl | 2 | 3 | 5 |
| FaSSIF | 2 | 5 | 8 |

### Example 4—Comparative Example—Pharmacokinetics in Dog of Compound 1 in Capsules and a Conventional Liquid Formulation

2×350 mg of Compound 1 filled in hard gelatin capsules was administered orally to Beagle Dogs in a PK study and compared to an orally administered solution of 20% hydroxypropylβcyclodextrin containing Compound 1. PK parameters following 50 mg/kg single oral administration to male Beagle Dogs:

| | | Nucleoside metabolite in plasma, n = 3 | | |
|---|---|---|---|---|
| | | AUC0-24 h | Cmax | tmax |
| po dose | Formulation | [μM*h] | [μM] | [h] |
| 90 μmol/kg, | 20% HPβCD | 135 ± 61 | 22 ± 16 | 2.3 ± 1.2 |
| 3 mL/kg | | | | |
| 2 × 350 mg | Powder in | 73 ± 49 | 7.8 ± 6.9 | 3.3 ± 1.2 |
| | capsule | | | |

### Example 5—Pharmacokinetics in Mouse of Compound 1 in an Ethanol-Free Embodiment of the Invention in Comparison to a Conventional Liquid Formulation

Exposure of plasma nucleoside after oral administration to mouse of Compound 1 in conventional polyethylene glycol formulations.

| | | Nucleoside metabolite in plasma | | |
|---|---|---|---|---|
| po dose | | AUC0-t | Cmax | tmax |
| [μmol/kg] | Formulation | [nmol*h/L] | [nM] | [h] |
| 480 | PEG400:water | 42295 | 10408 | 1.0 |
| | 50/50 | | | |
| | Conventional | | | |
| 480 | PEG400:Tween80 | 83440 | 22132 | 1.3 |
| | 95/5 | | | |
| | Invention | | | |

### Example 5. Solubility of Compound 1 in Ethanol

100.43 mg of Compound 1 was weighed into a glass vial. 37 μl of 99% ethanol was added so that final concentration was 2700 mg/ml. The formulation was heated to 50-60° C. during gentle agitation until the drug substance was fully dissolved. A transparent syrup was obtained and the solution remained transparent after cooling and storage at room temperature for more than 48 hours with no visible crystals.

### Example 6. Solubility of Compound 1 in Ethanol

200.26 mg of Compound 1 was weighed into a glass vial. 67 μl of 99% ethanol was added so that final concentration was 3000 mg/ml. The formulation was heated to 50-60° C. during gentle agitation until the drug substance was fully dissolved. A transparent syrup was obtained. After cooling and storage at room temperature, the solution solidified to a slightly opaque semisolid with no visible crystals

### Example 7. Formulation A 60 mg/ml

### Vehicle:

95% Ethanol, 7.5% v/v
PEG400, 87.5% v/v
Tween 80 5.0% v/v
PVP 20 mg/ml

Procedure for 7.5 mL of 60 mg/ml Compound 1:

1. Weigh 450.36 mg of Compound 1

2. Dissolve in 95% ethanol−7.5% v/v of 7.5 mL=0.56 mL.

3. Sonicate and warm until dissolved.

4. Add 6.56 mL of PEG400. Mix until a clear homogenous solution is obtained.

5. Add 0.38 mL of Tween 80. Mix until a clear homogenous solution is obtained.

6. Weigh 150.16 mg of polyvinylpyrrolidone and add to the above solution. Mix until a clear isotropic, homogenous solution is obtained.

Formulation A was physically stable at least 2 months observed by visual inspection with no visible phase separations or precipitates. An in vitro test was performed by diluting formulation A and B in a buffer of pH=2 with a dilution factor 10, 100 μl was diluted in 900 μl of the buffer. Smooth homogenous emulsions were obtained after gentle agitation. The emulsions were observed by light microscopy. Formulation A formed a macroemulsion with a droplet diameter less than 5 μm. The formulation was also measured by dynamic light scattering diluted in buffer 1:10 and the emulsion droplets hydrodynamic diameter was 1116 nm with a DynaPro Nanostar detector (Wyatt Technology).

Formulation A was orally administered to rats at two dose levels and showed the following pharmacokinetics:

Formulation A

| Dose [mg/kg] | AUClast [μM*h] | Cmax [μM] | Tmax [h] |
|---|---|---|---|
| 90 | 60 ± 17 | 6.8 ± 3.2 | 2.3 ± 1.2 |
| 300 | 100 ± 14 | 8.9 ± 2.1 | 3.7 ± 1.2 |

### Example 8. Formulation B 200 mg/ml

### Vehicle:

95% Ethanol, 7.5% v/v
PEG400, 62.5% v/v
Solutol HS15, 30% v/v
PVP 20 mg/ml

Procedure for 2 mL of 200 mg/ml:

1. Weigh 400.07 mg of Compound 1

2. Dissolve in 95% ethanol−7.5% v/v of 2 mL=0.15 mL

3. Sonicate and warm until dissolved.

4. Add 1.25 mL of PEG400. Mix until a clear homogenous solution is obtained.

5. Warm Solutol HS 15 gently until the semisolid is completely liquefied.

6. Add 0.6 mL Solutol HS 15. Mix until a clear homogenous solution is obtained.

7. Weigh 40.06 mg of polyvinylpyrrolidone and add to the above solution.

Mix until a clear isotropic, homogenous solution is obtained.

Formulation B was physically stable at least 2 months observed by visual inspection with no visible phase separations or precipitates. An in vitro test was performed by diluting Formulation B in a buffer of pH=2 with a dilution factor 10, 100 μl was diluted in 900 μl of the buffer. Smooth homogenous emulsions were obtained after gentle agitation. Formulation B formed a colloidal microemulsion with droplet sizes less than 1 μm measured by light microscopy at a magnification of 400. Also Brownian motion of submicron particles was observed indicating the colloidal nature of the emulsion. By dynamic light scattering a formulation B with 200 mg/ml of (1) diluted in buffer 1:10 the emulsion droplets had a hydrodynamic diameter of 542 nm with the DynaPro Nanostar detector (Wyatt Technology).

Formulation B was orally administered to rats at two dose levels and showed the following pharmacokinetics:

Formulation B

| Dose [mg/kg] | AUClast [μM*h] | Cmax [μM] | Tmax [h] |
|---|---|---|---|
| 90 | 60 ± 9.6 | 5.5 ± 1.8 | 3.7 ± 1.2 |
| 300 | 134 ± 35 | 11 ± 4.4 | 3.0 ± 0 |

### Example 9: Additional Formulations

The formulations in the table below were prepared substantially as shown in Examples 6 and 7.

| | | | | | | Emulsion quality |
|---|---|---|---|---|---|---|
| | 99% | Solutol | | Ratio | Concentration of | after aqueous |
| Compound 1 | EtOH | HS15 | PEG400 | drug:solutol | Compound 1 | dilution by visual |
| [mg] | [μl] | [μl] | [μl] | w/v % | [mg/ml] | inspection |
| 100 | 100 | 400 | — | 0.25 | 200 | OK |
| 52.5 | 25 | 150 | — | 0.35 | 300 | OK |
| 80 | 30 | 200 | — | 0.4 | 348 | OK |
| 200 | 75 | 300 | 625 | 0.66 | 200 | OK |

### Example 10: Formulation C, 200 mg/ml

| | | |
|---|---|---|
| Compound 1 | 200 | mg |
| 95% ethanol | 100 | μl |
| Solutol HS15 | 300 | μl |
| Capmul MCM | 300 | μl |
| Miglyol 812 | 300 | μl |

An isotropic concentrate was obtained after mixing, physically stable more than three months stored at room temperature. Upon aqueous dilution 10:1000 a smooth macroemulsion was obtained.

### Example 11: Formulation D, 50 mg/ml

| | | |
|---|---|---|
| Compound 1 | 50 | mg |
| PEG 400 | 100 | μl |
| Solutol HS15 | 300 | μl |
| Capmul MCM | 300 | μl |
| Miglyol 812 | 300 | μl |

An isotropic concentrate was obtained after mixing, physically stable more than three months stored at room temperature. Upon aqueous dilution 10:1000 a smooth microemulsion was obtained.

### Example 12: Formulation E, 200 mg/ml

| Compound 1 | 200 | mg |
|---|---|---|
| 95% ethanol | 75 | μl |
| Vit E TPGS (D-alpha-tocopheryl | 250μ | |
| polyethylene glycol 1000 succinate) | | |
| PEG 400 | 675 | μl |

An isotropic concentrate was obtained after mixing, physically stable more than three months at room temperature. Upon aqueous dilution 100:1000 a smooth colloidal microemulsion was obtained.

### Example 13: Ethanol-Free SEDDS 50 mg/ml

| | | |
|---|---|---|
| Compound 1 | 50 | mg |
| PEG 400 | 700 | μl |
| Solutol HS15 | 300 | μl |

This example exhibited physical stability less than one month at room temperature storage, phase separation and/or gelation, but this is believed to reflect instability of the solutol in the relatively low concentration of Compound 1. In particular, Formulation D above, which is also an ethanol-free composition was stable, presumably due to stabilization via the glyceride content. In ethanol-containing compositions where the concentration of compound 1 can be higher, solutol stability is readily achieved.

### Example 14: Porous Silica Gel Carrier

### Formulation

| Raw material | Amount (mg) | Supplier |
|---|---|---|
| Compound 1 | 350 | Medivir |
| Syloid XPD | 300 | Grace |
| Ethanol 95% | 262 μl | Kemetyl |
| Gelatin capsule 00El white | 130 | Capsugel |

### Manufacturing Method

Compound 1 was weighed into a glass vial.
Ethanol 95% was added to the glass vial with a pipette
Compound 1 was allowed to dissolve
Syloid XPD was added to the glass vial and slowly agitated with a spatula. The mix was left overnight
The mix was weighed into a capsule

### Example 15: Formula E

### Formulation

| Raw material | Amount (mg) | Supplier |
|---|---|---|
| Compound 1 | 350 | Medivir |
| Solutol HS-15 (Kolliphor HS15) | 500 μl | BASF |
| Ethanol 95% | 262 μl | Kemetyl |
| Gelatin capsule 00El white | 130 | Capsugel |

### Manufacturing Method

Compound 1 was weighed into a glass vial.
Ethanol 95% was added to the glass vial with a pipette
Compound 1 was allowed to dissolve
Solutol HS-15 was melted and added with pipette to the glass vial. The solution was agitated slowly with a spatula.
The mix was weighed into a capsule.

### Example 16: Ethanol Free Capsule (Comparative Example)

### Formulation

| Raw material | Amount (mg) | Supplier |
|---|---|---|
| Compound 1 | 350 | Medivir |
| Gelatin capsule 00El white | 130 | Capsugel |

### Manufacturing Method

350 mg Compound 1 was manually weighed into a capsule.

### Example 17: Release Rate Determination

Analytical Methods

Dissolution settings Apparatus 1 (basket) Erweka DT50:
Media 900 ml 0.1M HCl
Temp 37° C.
Stirring rate 100 rpm
Sampling volume 5.00 ml
Sampling time 20, 45 and 90 minutes
Filter 0.45 μm PP membranse

UPLC settings
Column: Acquity HSS PFP 50*2.1 mm, 1.8 μm
Mobile phase A: 5 mM ammonium acetate
Mobile phase B: acetonitrile
Gradient 0.60 ml/min:
0 min: 20% B, 2.50 min: 40% B, 2.60 min: 99% B, 3.00 min: 99% B, 3.10 min: 20% B
Injection volume 1 μl
Column temperature: 50° C.

Results

### Formulation of (Comparative) Example 16

| | Released % | | |
|---|---|---|---|
| Sample No | 20 min | 45 min | 90 min |
| 1 | 2.1 | 4.7 | 8.6 |
| 2 | 2.3 | 5.1 | 9.0 |
| mean | 2.2 | 4.9 | 8.8 |

### Formulation of Example 14 (Solid Carrier)

| | Released % | | |
|---|---|---|---|
| Sample No | 20 min | 45 min | 90 min |
| 1 | 48.6 | 60.3 | 66.6 |
| 2 | 51.3 | 61.9 | 67.3 |
| mean | 50.0 | 61.1 | 67.0 |

### Formulation of Example 15 (Formulation E)

| | Released % | | |
|---|---|---|---|
| Sample No | 20 min | 45 min | 90 min |
| 1 | 15.1 | 27.1 | 40.5 |
| 2 | 17.6 | 29.6 | 42.5 |
| mean | 16.4 | 28.4 | 41.7 |

It is clear from the above that the ethanol-containing formulation E had substantially better release than the active ingredient loaded direct into the capsule and that even better release was seen in an ethanolic solution of Compound 1 adsorbed on a solid carrier within the capsule.

1. A pharmaceutical composition comprising a compound of the formula I: 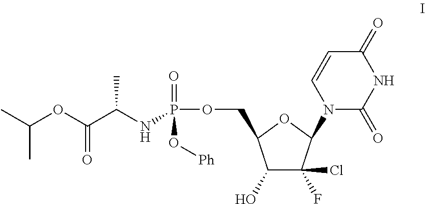 [SMILES: CC(C)OC(=O)[C@H](C)N[P@](=O)(OC[C@H]1O[C@@H](n2ccc(=O)[nH]c2=O)[C@@](F)(Cl)[C@@H]1O)Oc1ccccc1]
wherein the pharmaceutical composition further comprises ethanol.

2. A composition according to claim 1, wherein the composition further contains Solutol HS15.

3. A composition according to claim 1, wherein the compound of formula I is present in the range 150 to 3000 mg/ml in ethanol, preferably 250-2700 mg/ml.

4. A composition according to claim 1 in a pharmaceutically acceptable unit dosage form, comprising:
a) 200-750 mg of the compound of formula I;
b) 40-400 mg ethanol;
c) a nucleation inhibitor;
d) a hydrophilic surfactant with an HLB>12; and, optionally
e) triglyceride, diglyceride, monoglyceride or mixtures thereof; and/or:
f) a hydrophilic cosolvent selected from propylene glycol, polyethylene glycol, glycerol, 2-(2-ethoxyethoxy)ethanol, and mixtures thereof.

5. A composition according to claim 4, wherein the unit dosage form comprises 50-250 mg ethanol, preferably 59-222 mg.

6. A composition according to claim 4, wherein the nucleation inhibitor is a hydrophilic polymer selected from:
homopolymers of N-vinyl lactam,
copolymers of N-vinyl lactam,
cellulose esters,
cellulose ethers,
polyalkylene oxides,
polyacrylates,
polymethacrylates,
polyacrylamides,
polyvinyl alcohols,
vinyl acetate polymers,
oligosaccharides, or
polysaccharides.

7. A composition according to claim 6, wherein the hydrophilic polymer is selected from:
homopolymer of N-vinyl pyrrolidone,
copolymer of N-vinyl pyrrolidone,
copolymer of N-vinyl pyrrolidone and vinyl acetate,
copolymer of N-vinyl pyrrolidone and vinyl propionate,
graft copolymer of polyethylene glycol/polyvinyl caprolactam/polyvinyl acetate (e.g., Soluplus),
polyvinylpyrrolidone,
methylcellulose,
ethylcellulose,
hydroxyalkylcelluloses,
hydroxypropylcellulose,
hydroxyalkylalkylcellulose,
hydroxypropylmethylcellulose,
cellulose phthalate,
cellulose succinate,
cellulose acetate phthalate,
hydroxypropylmethylcellulose phthalate,
hydroxypropylmethylcellulose succinate,
hydroxypropylmethylcellulose acetate succinate,
polyethylene oxide,
polypropylene oxide,
copolymer of ethylene oxide and propylene oxide,
methacrylic acid/ethyl acrylate copolymer,
methacrylic acid/methyl methacrylate copolymer,
butyl methacrylate/2-dimethylaminoethyl methacrylate copolymer,
poly(hydroxyalkyl acrylate),
poly(hydroxyalkyl methacrylate),
copolymer of vinyl acetate and crotonic acid,
partially hydrolyzed polyvinyl acetate,
carrageenan,
galactomannan,
xanthan gum,
or a combination thereof.

8. A composition according to claim 7, wherein the nucleation inhibitor comprises polyvinylpyrrolidone.

9. A composition according to claim 4, wherein the hydrophilic surfactant comprises:
polyoxyethylene castor oil derivates,
mono fatty acid ester of polyoxyethylene sorbitan,
polyoxyethylene alkyl ether,
polyoxyethylene alkylaryl ether,
polyethylene glycol fatty acid ester,
alkylene glycol fatty acid mono ester,
sucrose fatty acid ester, or
sorbitan fatty acid mono ester.

10. A composition according to claim 9, wherein the hydrophilic surfactant comprises:
polyoxyethyleneglycerol triricinoleate,
polyoxyl 35 castor oil (Cremophor EL; BASF Corp.),
polyoxyethyleneglycerol oxystearate such as polyethylenglycol 40 hydrogenated castor oil (Cremophor RH 40, also known as polyoxyl 40 hydrogenated castor oil or macrogolglycerol hydroxystearate),
polyethylenglycol 60 hydrogenated castor oil (Cremophor RH 60),
mono fatty acid ester of polyoxyethylene sorbitan, such as mono fatty acid ester of polyoxyethylene (20) sorbitan, e.g. polyoxyethylene (20) sorbitan monooleate (Tween 80), polyoxyethylene (20) sorbitan monostearate (Tween 60), polyoxyethylene (20) sorbitan monopalmitate (Tween 40) or polyoxyethylene (20) sorbitan monolaurate (Tween 20),
polyoxyethylene (3) lauryl ether,
polyoxyethylene (5) cetyl ether,
polyoxyethylene (2) stearyl ether,
polyoxyethylene (5) stearyl ether,
polyoxyethylene (2) nonylphenyl ether,
polyoxyethylene (3) nonylphenyl ether,
polyoxyethylene (4) nonylphenyl ether,
polyoxyethylene (3) octylphenyl ether,
PEG-200 monolaurate,
PEG-200 dilaurate,
PEG-300 dilaurate,
PEG-400 dilaurate,
PEG-300 distearate,
PEG-300 dioleate,
propylene glycol monolaurate (e.g., lauroglycol FCC),
D-alpha-tocopheryl polyethylene glycol 1000 succinate,
sucrose monostearate,
sucrose distearate,
sucrose monolaurate,
sucrose dilaurate,
sorbitan mono laurate,
sorbitan monooleate,
sorbitan monopalnitate,
sorbitan stearate,
or a combination thereof.

11. A composition according to claim 10, wherein the surfactant comprises Solutol HS15.

12. A composition according to claim 4, wherein the hydrophilic cosolvent comprises polyethylene glycol 400.

13. A composition according to claim 4, wherein the glyceride is Capmul MCM.

14. A composition according to claim 1, adsorbed on an inorganic mesoporous carrier with a specific surface area 100-1000 m²/g, preferably 100-800 m²/g.

15. A composition according to claim 14, wherein the carrier is selected from aerosil, neusilin, CaCO₃, MgCO₃ and mixtures thereof.

16. A composition according to claim 4 encapsulated in a hard shell or preferably softgel capsule.

17. A composition according to claim 4, wherein the dosage unit contains 340-580 mg Compound 1.

18. A pharmaceutical composition comprising a compound of the formula I: 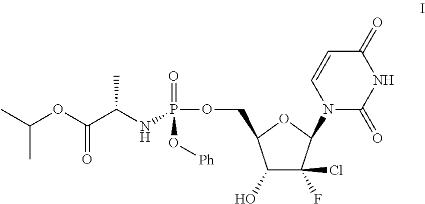 [SMILES: CC(C)OC(=O)[C@H](C)N[P@](=O)(OC[C@H]1O[C@@H](n2ccc(=O)[nH]c2=O)[C@@](F)(Cl)[C@@H]1O)Oc1ccccc1]
in a pharmaceutically acceptable vehicle comprising w:w
a) 0-80% trigyceride, diglyceride, monoglyceride or mixtures thereof,
b) 0-50% hydrophilic cosolvent selected from ethanol, propylene glycol, polyethylene glycol, and mixtures thereof;
c) nucleation inhibitor; and
d) hydrophilic surfactant with an HLB>12;
wherein the % w:w ratio of a:b:c:d is selected from the table below and totals 100:

| SEDDS form | a) | b) | c) | d) |
|---|---|---|---|---|
| Pouton | 40-80 | 0-40 | 0.01-10 | 20-40 |
| Type IIIA | | | | |
| Pouton | <20 | 20-50 | 0.01-10 | 20-50 |
| Type IIIB | | | | |
| Pouton | — | 0-50 | 0-10 | 30-95 |
| Type IV | | | | |

19. A pharmaceutical composition according to claim 18, wherein the compound of formula I is at least 90%, preferably at least 95%, enantiomerically pure as regards the P(S) diastereomer.

20. A pharmaceutical composition according to claim 18, wherein the nucleation inhibitor is a hydrophilic polymer selected from:
homopolymers of N-vinyl lactam,
copolymers of N-vinyl lactam,
cellulose esters,
cellulose ethers,
polyalkylene oxides,
polyacrylates,
polymethacrylates,
polyacrylamides,
polyvinyl alcohols,
vinyl acetate polymers,
oligosaccharides, or
polysaccharides.

21. A pharmaceutical composition according to claim 20, wherein the hydrophilic polymer is selected from
homopolymer of N-vinyl pyrrolidone,
copolymer of N-vinyl pyrrolidone,
copolymer of N-vinyl pyrrolidone and vinyl acetate,
copolymer of N-vinyl pyrrolidone and vinyl propionate,
graft copolymer of polyethylene glycol/polyvinyl caprolactam/polyvinyl acetate (e.g., Soluplus),
polyvinylpyrrolidone,
methylcellulose,
ethylcellulose,
hydroxyalkylcelluloses,
hydroxypropylcellulose,
hydroxyalkylalkylcellulose,
hydroxypropylmethylcellulose,
cellulose phthalate,
cellulose succinate,
cellulose acetate phthalate,
hydroxypropylmethylcellulose phthalate,
hydroxypropylmethylcellulose succinate,
hydroxypropylmethylcellulose acetate succinate,
polyethylene oxide,
polypropylene oxide,
copolymer of ethylene oxide and propylene oxide,
methacrylic acid/ethyl acrylate copolymer,
methacrylic acid/methyl methacrylate copolymer,
butyl methacrylate/2-dimethylaminoethyl methacrylate copolymer,
poly(hydroxyalkyl acrylate),
poly(hydroxyalkyl methacrylate),
copolymer of vinyl acetate and crotonic acid,
partially hydrolyzed polyvinyl acetate,
carrageenan,
galactomannan,
xanthan gum,
or a combination thereof.

22. A pharmaceutical composition according to claim 18, wherein the hydrophilic surfactant comprises:
polyoxyethylene castor oil derivates,
mono fatty acid ester of polyoxyethylene sorbitan,
polyoxyethylene alkyl ether,
polyoxyethylene alkylaryl ether,
polyethylene glycol fatty acid ester,
alkylene glycol fatty acid mono ester,
sucrose fatty acid ester, or
sorbitan fatty acid mono ester.

23. A pharmaceutical composition according to claim 22, wherein the hydrophilic surfactant comprises:
polyoxyethyleneglycerol triricinoleate;
polyoxyl 35 castor oil (Cremophor EL; BASF Corp.),
polyoxyethyleneglycerol oxystearate such as polyethylenglycol 40
hydrogenated castor oil (Cremophor RH 40, also known as polyoxyl 40
hydrogenated castor oil or macrogolglycerol hydroxystearate),
polyethylenglycol 60 hydrogenated castor oil (Cremophor RH 60),
mono fatty acid ester of polyoxyethylene sorbitan, such as mono fatty acid ester of polyoxyethylene (20) sorbitan, e.g. polyoxyethylene (20) sorbitan monooleate (Tween 80), polyoxyethylene (20) sorbitan monostearate (Tween 60), polyoxyethylene (20) sorbitan monopalmitate (Tween 40) or polyoxyethylene (20) sorbitan monolaurate (Tween 20),
polyoxyethylene (3) lauryl ether,
polyoxyethylene (5) cetyl ether,
polyoxyethylene (2) stearyl ether,
polyoxyethylene (5) stearyl ether,
polyoxyethylene (2) nonylphenyl ether,
polyoxyethylene (3) nonylphenyl ether,
polyoxyethylene (4) nonylphenyl ether,
polyoxyethylene (3) octylphenyl ether,
PEG-200 monolaurate,
PEG-200 dilaurate,
PEG-300 dilaurate,
PEG-400 dilaurate,
PEG-300 distearate,
PEG-300 dioleate,
propylene glycol monolaurate (e.g., lauroglycol FCC),
D-alpha-tocopheryl polyethylene glycol 1000 succinate,
sucrose monostearate,
sucrose distearate,
sucrose monolaurate,
sucrose dilaurate,
sorbitan mono laurate,
sorbitan monooleate,
sorbitan monopalnitate,
sorbitan stearate,
or a combination thereof

24. A pharmaceutical composition according to claim 23, wherein the surfactant comprises Solutol HS15.

25. A pharmaceutical composition according to claim 18, wherein the hydrophilic cosolvent comprises polyethylene glycol 400.

26. A pharmaceutical composition according to claim 18, wherein the hydrophilic cosolvent comprises ethanol.

27. A pharmaceutical composition according to claim 26, wherein the compound of formula 1 is present in the range 150 to 3000 mg/ml ethanol, preferably 250-2700 mg/ml.

28. A pharmaceutical composition according to claim 26, in unit dosage form and comprising 200-750 mg of the compound of formula I and 40-400 mg ethanol;

29. A pharmaceutical composition according to claim 28, wherein the unit dosage form comprises 50-250 mg ethanol, preferably 59-222 mg.

30. A pharmaceutical composition according to claim 18, wherein the unit dosage form contains 340-580 mg Compound 1.

31. A pharmaceutical composition according to claim 18, wherein the glyceride comprises Capmul MCM.

32. A pharmaceutical composition according to claim 18, adsorbed on an inorganic mesoporous carrier a specific surface area 100-1000 m²/g, preferably 100-800 m²/g.

33. A pharmaceutical composition according to claim 32, wherein the carrier is selected from aerosil, neosilin, CaCO₃, MgCO₃ and mixtures thereof.

34. A pharmaceutical composition according to claim 18, in unit dosage form and encapsulated in a hard shell or preferably a softgel capsule.
